(19) United States
(12) Patent Application Publication
(10) Pub. No.: US 2018/0103858 A1
(43) Pub. Date: Apr. 19, 2018

(54) EARBUD ELECTROCARDIOGRAM MONITOR AND ASSOCIATED SYSTEMS AND METHODS

(71) Applicant: Medicomp, Inc., Melbourne, FL (US)

(72) Inventors: Sean Marcus, Rockledge, FL (US); Anthony Balda, Satellite Beach, FL (US); George Koos, Melbourne Beach, FL (US); Scott Baskerville, Melbourne, FL (US)

(73) Assignee: Medicomp, Inc., Melbourne, FL (US)

(21) Appl. No.: 15/556,497

(22) PCT Filed: Mar. 14, 2016
(86) PCT No.: PCT/US16/22311

§ 371 Date: Sep. 7, 2017

Related U.S. Application Data
(60) Provisional application No. 62/132,221, filed on Mar. 12, 2015.

(51) Int. Cl. A61B 5/024; A61B 5/0205; A61B 5/046

(52) U.S. Cl. CPC A61B 5/02438; A61B 5/0205; A61B 5/11; A61B 5/02405; A61B 5/046

(57) ABSTRACT

A method of monitoring a health status of a patient using a monitoring system comprising a processor, an electrical signal conversion unit, and a pair of earbud electrocardiogram (ECG) monitors. Each earbud monitor comprises conductive electrode component (e.g., physiological-type sensor) configured to receive biopotential signals from a respective ear of the patient. The electrical signal conversion unit controls earbud operations, and converts earbud readings into ECG data that are transmitted to a smartphone for further analysis. The electrical signal conversion unit components may include a System on a Chip (SoC) comprising a data store, a processor, and a power supply.

### RELATED APPLICATIONS

This application claims the benefit under 35 U.S.C. § 119(a)-(d) of International Publication Number WO 2016/145438 filed on Mar. 14, 2016 and titled Earbud Electrocardiogram Monitor and Associated Systems and Methods, which claims the benefit under 35 U.S.C. § 119(e) of U.S. Provisional Patent Application Ser. No. 62/132,221 filed by the inventor of the present application on Mar. 12, 2015, and titled Earbud Electrocardiogram Monitor and Associated Systems and Methods, the entire content of which is incorporated herein by reference except to the extent that disclosure therein is inconsistent with disclosure herein.

### FIELD OF THE INVENTION

The present invention relates generally to electrocardiogram (ECG) monitoring and, more particularly, to wearable devices with integrated ECG sensors for ambulatory ECG monitoring, and related systems and methods.

### BACKGROUND

Heart disease is a leading cause of death in the United States. Some patients may benefit from long-term ECG monitoring outside of a clinical setting. For example, atrial fibrillation and myocardial ischemia may occur episodically. Some episodes may occur without patient symptoms. Myocardial ischemia, if persistent and serious, can lead to a myocardial infarction (heart attack). During a myocardial infarction, electrophysiological changes may be detected by an ECG. For accurate diagnosis and effective treatment of many episodic heart conditions, medical professionals need to receive accurate and timely information regarding the frequency and duration of such episodes.

In conventional long-term ECG monitoring, such as with continuous Holter monitors or event monitors, mounting of the monitor typically involves preparation of the patient's skin to receive the monitoring device. Chest hair may be shaved or clipped from men. The skin is abraded to remove dead skin cells, and cleaned. A technician trained in electrode placement applies the electrodes to the skin with an adhesive. Each electrode of such conventional monitors is attached to an insulated wire that is routed some distance across the patient's body to an amplifier designed to amplify the ECG signal in preparation for further processing. Such monitoring systems are often worn by a patient for up to a month.

Traditional long-term monitoring systems like those described above present a number of problems. For example, abrading in preparation for electrode mounting often leaves the patient's skin irritated. During use, the patient must be careful not to pull on the wires connected to the electrodes, lest the electrodes be pulled off the skin. Removing an electrode with its strong adhesive may be painful to the patient. Furthermore, certain types of electrodes require use of a gel next to the skin to improve conductivity at the point of connection of the metal electrode to the skin. Prolonged exposure to the gel can irritate the skin. These and other discomfort factors associated with traditional long-term monitoring solutions may discourage a patient from using the ECG monitor as directed by medical personnel.

Alternative health monitoring system designs exist that attempt to address the many shortcomings of traditional ECG monitors. For example, some monitor implementations known in the art are based on an article of apparel designed to be conveniently and comfortably worn by the patient, such as a wrist band or earbud. However, the still-prominent profile of such monitors still may cause wear, and use of such devices can be uncomfortable and error-prone. Also for example, some monitors are implemented as earphones equipped with sensors and data communications means, such as the following.

U.S. Pat. No. 7,769,435 to Kuo et al. discloses an earphone sensor system for measuring electrocardiogram signals. The electrocardiogram signal analyzing apparatus includes an amplifier module, a microcontroller, a display, a radio module and a housing having conductive contacts. The earphone sensor system can be associated with commercial gadgets and used for musical treatments and bio-feedback.

W.O. Patent Application No. 2009069037 by Powers discloses a heart monitor including an electroacoustic transducer such as an earphone coupled to a controller. The transducer is positioned in a person's ear such that signals from the transducer are processed to determine the presence of pulsatile blood flow. The heart monitor may be incorporated into a portable media playback device alternating between playback and monitoring mode or performing both simultaneously using one earphone for each function.

U.S. Patent Application No. 2013/0158423 by Kapoor is directed to a system for acquiring an electrical footprint of the heart, electrocardiogram (ECG) and heart rate variability monitoring, incorporated into a mobile device accessory. The ECG signal is acquired and transmitted to a server via the mobile device, offering accurate heart rate variability biofeedback measurement which is portable and comfortable during normal daily life.

The implementations described above, as well as similar devices, systems, and methods known in the art, may require some special modification to a smartphone or other mobile device for proper function, are limited to one channel, and/or require data transmission to an external server for signal analysis. Consequently, a need exists for increasingly comfortable and convenient monitoring devices for both personal and medical use, and that overcome the shortcomings of common implementations in the field.

While certain aspects of conventional technologies have been discussed to facilitate disclosure of the invention, the applicant in no way disclaims these technical aspects, and it is contemplated that the claimed invention may encompass one or more of the conventional technical aspects discussed herein. The present invention may address one or more of the problems and deficiencies of the current availability and prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein, or limited to the particular embodiment for the invention used to illustrate the steps and functionality of the herein.

This background information is provided to reveal information believed by the applicant to be of possible relevance to the present invention. No admission is necessarily intended, nor should be construed, that any of the preceding information constitutes prior art against the present invention. This reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### SUMMARY OF THE INVENTION

With the foregoing in mind, embodiments of the present invention are related to systems and methods for monitoring a health status of a patient using a monitoring system. More specifically, the present invention may include a processor, an electrical signal conversion unit, and a pair of earbud electrocardiogram (ECG) monitors each including a respective conductive electrode component. The electrical signal conversion unit may carry a third electrode. Each conductive electrode component and the third electrode may comprise a physiological-type sensor.

A method aspect of the present invention may include the steps of positioning each of the conductive electrode components of the earbud ECG monitors in a respective ear of the patient, and receiving biopotential signals from each ear of the patient. The method further may include receiving, using the third electrode, biopotential signals from one of a left arm and a hip of the patient. Triggering of biopotential signal readings from all sensors may be by virtue of a record button carried by the electrical signal conversion unit.

Another method aspect of the present invention may include the steps of converting, using the electrical signal conversion unit, one or more of the biopotential signals described above into converted ECG data. The method further may include forwarding the converted ECG data to a smartphone, and analyzing the converted ECG data for cardiovascular health indicators using some number of smartphone applications. The earbud ECG monitors may advantageously cooperate with the electrical signal conversion unit to convert audio signals transmitted by the smartphone into sound using speaker units carried by the earbud ECG monitor.

More specifically, the electrical signal conversion unit may include a system on a chip (SoC) having a data store and a processor. The processor may retrieve processing functions from the data store and may execute those processing functions to identify the health status of the patient from the biopotential signals described above. The SoC may be configured in data communication, either wired or wirelessly, with the smartphone.

### BRIEF DESCRIPTION OF THE DRAWINGS

FIG. 1 is a schematic diagram of an earbud ECG monitor system according to an embodiment of the present invention.

FIGS. 2A and 2B are schematic diagrams of front and side elevation views, respectively, of an earbud monitor as used in connection with an earbud ECG monitor system according to an embodiment of the present invention.

FIG. 3 is a schematic diagram of a front elevation view of a signal conversion unit as used in connection with an earbud ECG monitor system according to an embodiment of the present invention.

FIG. 4 is a schematic diagram of perspective views of a headphone jack and a Y-joint cable, respectively, as used in connection with an earbud ECG monitor system according to an embodiment of the present invention.

FIG. 5 is a flow chart illustrating a method of ECG data analysis and display as used in connection with an earbud ECG monitor system according to an embodiment of the present invention.

FIG. 6 is a block diagram of a system on a chip (SoC) as used in connection with an earbud ECG monitor system according to an embodiment of the present invention.

FIG. 7 a block diagram representation of a machine in the example form of a computer system according to an embodiment of the present invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

Throughout this disclosure, the present invention may be referred to as an earbud ECG system, an earbud system, an ECG monitor system, an ECG system, a heartrate monitor system, an earbud heartrate monitor, an earbud, a monitor, a computer program product, a computer program, a product, a system, a device, and a method. Furthermore, the present invention may be referred to as relating to generally to biometric monitoring. Those skilled in the art will appreciate that this terminology does not affect the scope of the invention.

Example methods and systems for an earbud ECG monitor system are described herein below. In the following description, for purposes of explanation, numerous specific details are set forth to provide a thorough understanding of example embodiments. It will be evident, however, to one of ordinary skill in the art that the present invention may be practiced without these specific details and/or with different combinations of the details than are given here. Thus, specific embodiments are given for the purpose of simplified explanation and not limitation.

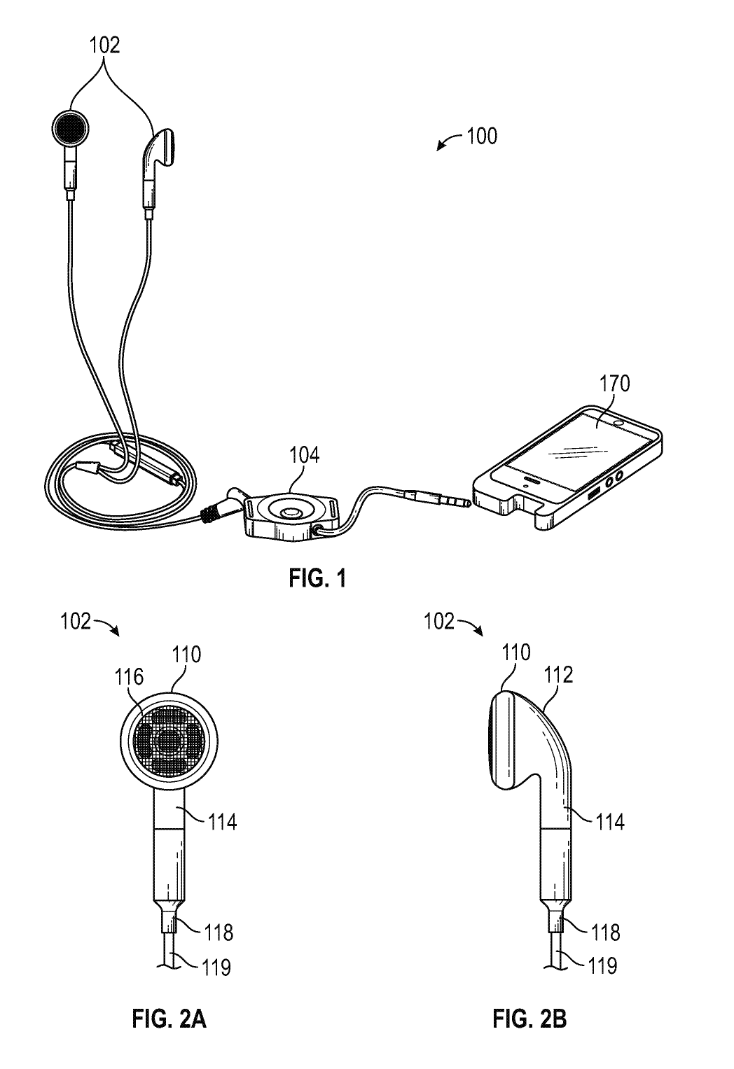

An embodiment of the invention, as shown and described by the various figures and accompanying text, provides an earbud electrocardiogram (ECG) monitor system 100 comprising some number of electrode-employing components 102 configured in electrical communication with an electrical signal conversion unit 104 which, in turn, may be configured in data communication with a processor 170 (as illustrated in FIG. 1). The earbud ECG monitor systems and associated methods 100 may advantageously allow users to easily monitor their cardiovascular health status when used in combination with a compatible processor such as, for example, and without limitation, a smartphone 170 and accompanying smartphone software application, without creating significant physical discomfort nor undue operational complexity for the user.

The related systems and methods of using the disclosed earbud ECG monitor 100 may advantageously leverage user skills that may be common among many individuals (e.g. devices carried in armbands in order to play music during exercise). These disclosed devices, systems, and methods may advantageously allow for the addition of ECG monitoring functionality without a significant increase in hardware requirements. Also, the disclosed system 100 advantageously may not require modification of the user's smartphone 170 (nor the purchase of a new smartphone), may allow for a consistently reliable hard-wired connection (e.g., may minimize connection failures common to wireless capability, as well as minimizing signal noise), and may maintain audio playback while offering comfort and efficiency in ECG measurement. For example, and without limitation, the present invention may be applied in the fields of cardiovascular medicine and health, sports medicine, and therapy in the area of ambulatory ECG monitoring. More specifically, the present invention may appeal to the fitness-oriented consumer and may also be applied to ECG-related procedures to drive patient compliance.

Referring now to FIGS. 2A and 2B, and continuing to refer to FIG. 1, each of the electrode-employing components 102 may be defined as comprising two specially-designed intra-concha earbuds similar to those used for audio playback in many smartphones and other media players. For example, and without limitation, an earbud 102 may include a standard speaker unit (not shown), a covering 116 for the speaker unit, and a conductive electrode component 110. The speaker unit may be configured to playback audio. The covering 116 for the speaker unit may be designed to be waterproof/sweat-proof. The electrode component 110 may be positioned on or around a casing 112 in a location conducive to biometric signal harvesting. The casing 112 may be configured to carry each of the aforementioned components, as well as a wire stem 114 (also referred to as an earbud stem) that may be configured to support a wire 119 as the wire 119 leaves the earbud 102. The wire stem 114 may advantageously prevent damage to the wire 119 or separation of the wire 119 from the earbud 102.

In one embodiment, the electrode/sensor component 110 may comprise a material that is both an excellent conductor and that may also be efficient in maintaining the earbud's 102 position within the concha of a human ear. For example, and without limitation, such materials may include electrically conductive silicone, which may easily be molded into a variety of shapes and which may allow for efficient design to increase signal quality. Also for example, and without limitation, the earbud 102 may be characterized by a rubber (or similar material) wire seal/guard 118 that may be positioned at the distal end of the earbud stem 114 to further protect the wiring 118 and to prevent damage due to moisture leakage on the inside of the device casing 112.

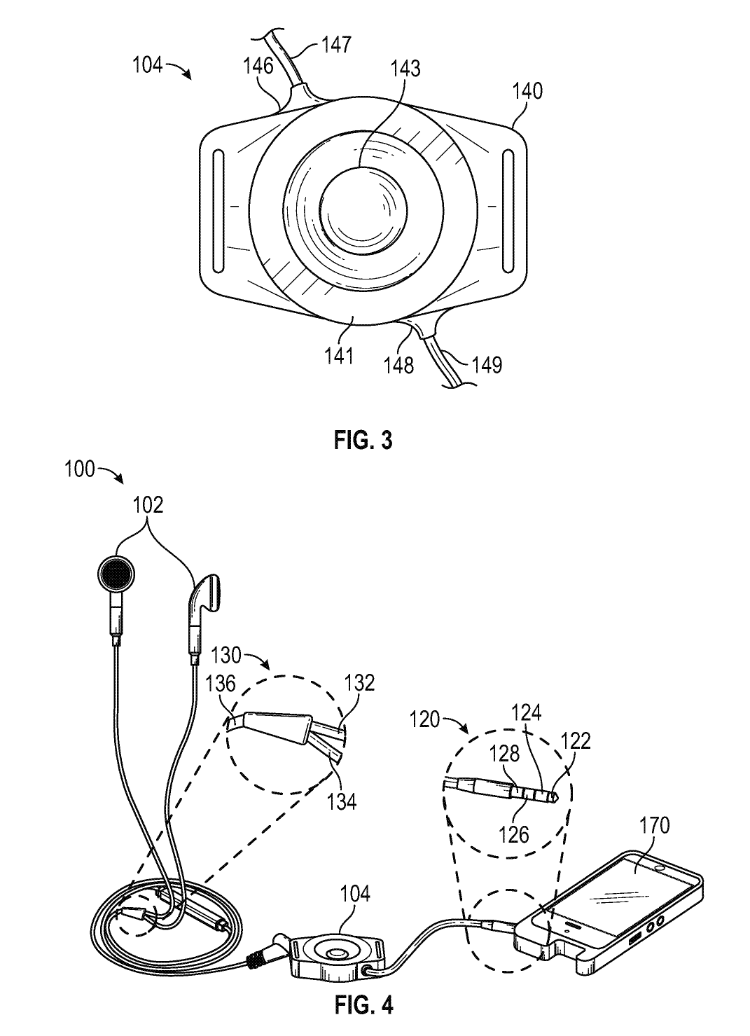

Referring now additionally to FIG. 3, and continuing to refer to FIG. 1, the signal conversion unit 104 may comprise circuitry configured to convert an ECG signal harvested by, for example, and without limitation, an earbud 102 into a digitized signal that may be transmitted to and recognized by a typical smartphone's 170 existing microphone hardware and software. The conversion unit 104, which may be implemented as a system on a chip (SoC), as described in more detail below, may include required circuit hardware/firmware/software, a casing 140, and entry 146 and exit 148 points for earbud cables 147, 149, respectively. For example, and without limitation, certain embodiments of the conversion unit 104 may include various interface elements meant for a user to perform a specific task (for example, and without limitation, a button that may trigger recording of a certain ECG segment, and/or a slide switch that may control music volume), various structures that may be designed to mechanically mount the conversion unit to the user's body (for example, and without limitation, an armband, an/or a belt clip), and a housing 141 that may be configured to carry a third electrode 143 that may be exposed at a position on the casing 140 where the electrode 143 may be in contact with the user's skin. For example, and without limitation, the casing 140 may also be designed to function as a mobile-device armband, where the mobile device being used may be fixed to a conversion unit 104 for the sole purpose of added convenience to the user.

In yet another embodiment of the present invention, the system 100 that uses the earbud ECG monitor 102 described above may also comprise an input apparatus alternative to the proposed modified earphones that may include a similar (if not same) conversion unit, wherein the earbuds may be replaced with regular ECG skin electrodes at the terminal end of the cable. These electrodes may be placed on the user's chest in an arrangement conducive to collecting viable ECG signal data. For example, and without limitation, the alternate system may include electrodes that may be able to support any desired number of leads.

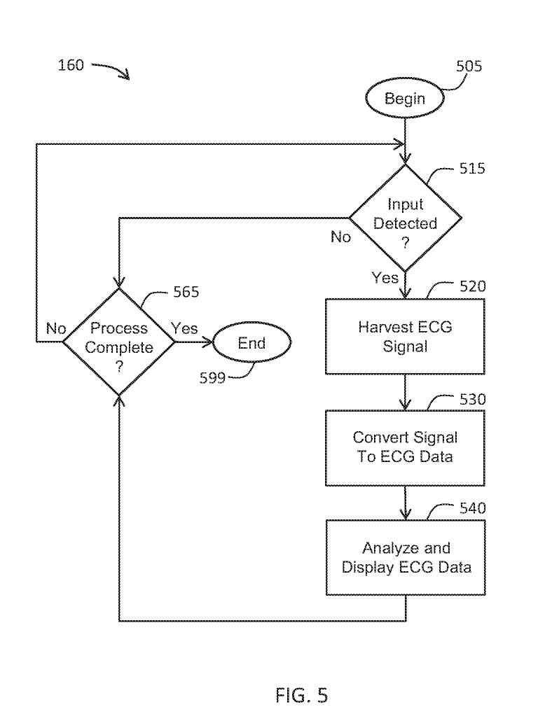

Referring now additionally to FIG. 4, and continuing to refer to FIG. 1, for example, and without limitation, a data connectivity mechanism between the conversion unit 104 and the smartphone 170 of the earbud ECG monitor system 100 may also include a standard TRRS-type Apple-compatible headphone jack 120 that may support both left 122 and right 124 stereo sound, as well as a microphone 128. A person of skill in the art will recognize that connector configurations may differ among mobile manufacturers. The system 100 so configured may advantageously allow the user to listen to audio from a smartphone device 170 as well as allow the device 170 to receive the converted ECG/microphone signal from the earbud ECG monitor's cable 149.

Referring again to FIGS. 2 and 3, and continuing to refer to FIGS. 1 and 4, wires 132, 134 from more than one earbud ECG monitor 119 may join each other to form one observable cable 136 to define a Y-joint 130 consistent with many existing headphone devices. The post-Y-joint 136 cable may then enter the signal conversion unit 104 at an input cable 147, and then may exit the unit 104 through an output cable 149 after signal processing. The post-conversion unit earbud cable may then enter (and end) at the TRRS-type manufacturer-compatible headphone jack 120, which may then be plugged in to the user's smartphone 170 or other compatible mobile device.

Referring now additionally to FIG. 5, and continuing to refer to FIG. 1, a computer-implemented method aspect of the present invention may use the earbud ECG monitor 102 described above to transmit harvested ECG data to the smartphone 170, the latter of which may include some type of mobile application capable of receiving, interpreting, analyzing, and displaying the harvested and converted signals delivered by the rest of the system. For example, and without limitation, this application may include functions for recording and saving segments of ECG data, providing important analytical and statistical data to the user relevant to health and fitness, and transferring the data if necessary (for example, and without limitation, via a mobile network, Wi-Fi, USB, or other means suitable for transferring data as may be understood by those skilled in the art). The application may also be able to function properly while running in the background if the user is multi-tasking. Standard music or other media audio playback may be unaffected by this system.

The overall function and order of systemic events, as illustrated in schematic 160, according to certain embodiments of the present invention, may be summarized as follows:

1) From the start (Block 505), a detected input signal (Block 515) may be harvested (Block 520) either from the two earbuds solely, from the electrodes 110, from the earbuds 102 and backside of conversion unit casing 143, or from any other viable combination of conductive components.

2) The signal may be relayed to the conversion unit 104 through the earbud/electrode cable 119, 130, where the signal may be processed and converted into a readable format of microphone data (Block 530), similar to existing TTM function, for example, and without limitation.

3) The newly converted ECG-Mic data may then be sent from the conversion unit 104 to the smartphone 170 through the cable 149 and/or the TRRS-type manufacturer-compatible headphone jack 120.

4) The ECG-Mic data may then be read by the smartphone 170/mobile device (either directly by the intended application, or by the phone itself and then pulled into the application). With the aforementioned electrode configuration (each ear, and then either left arm or hip), there may be two available channels of ECG data with the 3-lead setup.

5) At Block 540, analysis, transmission, or other subsequent processing of the ECG data may then be performed by the application and smartphone 170.

6) At Block 565, user and/or system input may dictate whether ECG signal harvesting and processing is complete. If so, the process may end at Block 599. If not, the process may seek to detect additional ECG signal input (Block 515).

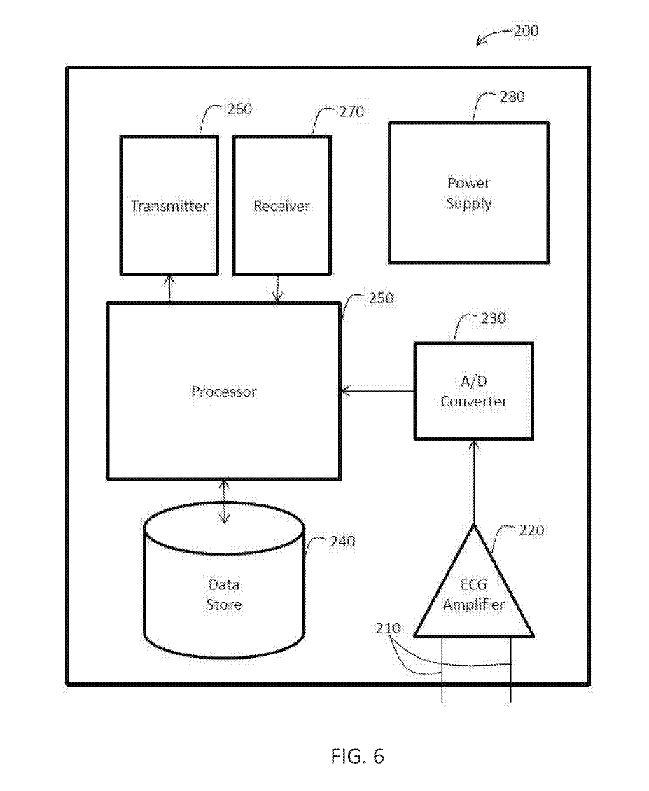

Referring now to FIG. 6, the SoC 200 component of the conversion unit 104 is now described in detail. For example, and without limitation, the SoC 200 may include at least one input connector 210 that may be connected to a signal amplifier 220. The signal amplifier 220 may come into contact with the conductor of the earbud 110, or may otherwise be in electrical communication with the conductor 110, to create an ECG lead. For example, and without limitation, the amplifier 220 may receive signals from the conductor 110 via an integrated wiring system. The signals from the conductor 110 may be amplified and subsequently converted by an analog-to-digital (A/D) converter 230. For example, and without limitation, the A/D converter 230 may be configured to digitize the signals from the amplifier 220, and may optionally include filters to filter the signals or perform signal processing and identification of physiological conditions. The amplified and converted signals may be directed into processing and storage circuitry that may include a data store 240 and a processor 250 to implement filtering and processing functions to provide intermediate results and to store information before transmission to computing resources 170 outside of the signal conversion unit 104. More specifically, the pre-processing circuitry of the SoC 200 may electrically couple the processed signals to a transmitter 260 (which may include an antenna) that may transmit the signals to the exterior system 170. The signals may be transmitted using, for example, Zigbee or Bluetooth protocols, to an exterior system that may be a computer, personal digital assistant (FDA), or wireless phone 170. Other circuitry (not shown) may include timing and interface circuitry.

As related above, the electrical conductor 110 may be in data communication with the data store 240, which may retain recorded signals until transmitted (transient) and/or may retain recorded signals until either manually or automatically deleted (persistent). The transmitter 260 may be configured to receive data from at least one of the conductor 110 and the data store 240, and to communicate the data representing electrical signals detected by the conductor 110. Also for example, and without limitation, the SoC 200 may comprise a receiver 270 in electrical communication with the data store 240. The receiver 270 may be configured to receive data and route those data to the data store 240 through the processor 250. For example, and without limitation, both communication of data from the transmitter 260 and receipt of data by the receiver 270 may occur wirelessly or over telephone lines. In one embodiment of wireless communication, the transmitter 260 and/or the receiver 270 may be implemented using radio frequency identification (RFID) technology. In other embodiments, the transmitter 260 and the receiver 270 may be provided in combination by a transceiver.

Continuing to refer to FIG. 6, the SoC 200 may also include a power supply 280 in electrical communication with at least one of the transmitter 260, the receiver 270, the processor 250, and the data store 240. In one embodiment, the power supply 280 may be a thin-film thermoelectric power generator configured to harvest, store, and channel electrical energy produced as body heat by subcutaneous tissue of the patient. In an alternative embodiment, the power supply 280 may harvest kinetic energy resulting from patient-initiated motion. In yet another alternative embodiment, the power supply 280 may comprise a resonance transformer for receiving near field wireless transmission of electrical energy (e.g., resonant inductive coupling). In still other embodiments, the power supply 280 may be provided by a battery.

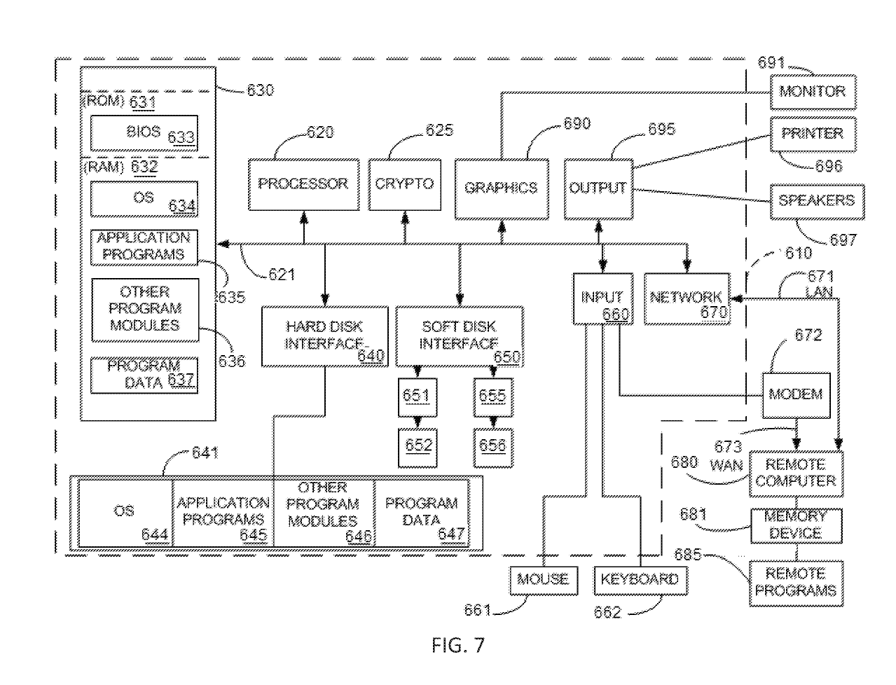

A skilled artisan will note that one or more of the aspects of the present invention may be performed on a computing device. The skilled artisan will also note that a computing device may be understood to be any device having a processor, memory unit, input, and output. This may include, but is not intended to be limited to, cellular phones, smart phones, tablet computers, laptop computers, desktop computers, personal digital assistants, etc. FIG. 7 illustrates a model computing device in the form of a computer 610, which is capable of performing one or more computer-implemented steps in practicing the method aspects of the present invention. Components of the computer 610 may include, but are not limited to, a processing unit 620, a system memory 630, and a system bus 621 that couples various system components including the system memory to the processing unit 620. The system bus 621 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI).

The computer 610 may also include a cryptographic unit 625. Briefly, the cryptographic unit 625 has a calculation function that may be used to verify digital signatures, calculate hashes, digitally sign hash values, and encrypt or decrypt data. The cryptographic unit 625 may also have a protected memory for storing keys and other secret data. In other embodiments, the functions of the cryptographic unit may be instantiated in software and run via the operating system.

A computer 610 typically includes a variety of computer readable media. Computer readable media can be any available media that can be accessed by a computer 610 and includes both volatile and nonvolatile media, removable and non-removable media. By way of example, and not limitation, computer readable media may include computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, FLASH memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by a computer 610. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared and other wireless media. Combinations of any of the above should also be included within the scope of computer readable media.

The system memory 630 includes computer storage media in the form of volatile and/or nonvolatile memory such as read only memory (ROM) 631 and random access memory (RAM) 632. A basic input/output system 633 (BIOS), containing the basic routines that help to transfer information between elements within computer 610, such as during start-up, is typically stored in ROM 631. RAM 632 typically contains data and/or program modules that are immediately accessible to and/or presently being operated on by processing unit 620. By way of example, and not limitation, FIG. 7 illustrates an operating system (OS) 634, application programs 635, other program modules 636, and program data 637.

The computer 610 may also include other removable/non-removable, volatile/nonvolatile computer storage media. By way of example only, FIG. 7 illustrates a hard disk drive 641 that reads from or writes to non-removable, nonvolatile magnetic media, a magnetic disk drive 651 that reads from or writes to a removable, nonvolatile magnetic disk 652, and an optical disk drive 655 that reads from or writes to a removable, nonvolatile optical disk 656 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 641 is typically connected to the system bus 621 through a non-removable memory interface such as interface 640, and magnetic disk drive 651 and optical disk drive 655 are typically connected to the system bus 621 by a removable memory interface, such as interface 650.

The drives, and their associated computer storage media discussed above and illustrated in FIG. 7, provide storage of computer readable instructions, data structures, program modules and other data for the computer 610. In FIG. 7, for example, hard disk drive 641 is illustrated as storing an OS 644, application programs 645, other program modules 646, and program data 647. Note that these components can either be the same as or different from OS 633, application programs 633, other program modules 636, and program data 637. The OS 644, application programs 645, other program modules 646, and program data 647 are given different numbers here to illustrate that, at a minimum, they may be different copies. A user may enter commands and information into the computer 610 through input devices such as a keyboard 662 and cursor control device 661, commonly referred to as a mouse, trackball or touch pad. Other input devices (not shown) may include a microphone, joystick, game pad, satellite dish, scanner, or the like. These and other input devices are often connected to the processing unit 620 through a user input interface 660 that is coupled to the system bus, but may be connected by other interface and bus structures, such as a parallel port, game port or a universal serial bus (USB). A monitor 691 or other type of display device is also connected to the system bus 621 via an interface, such as a graphics controller 690. In addition to the monitor, computers may also include other peripheral output devices such as speakers 697 and printer 696, which may be connected through an output peripheral interface 695.

The computer 610 may operate in a networked environment using logical connections to one or more remote computers, such as a remote computer 680. The remote computer 680 may be a personal computer, a server, a router, a network PC, a peer device or other common network node, and typically includes many or all of the elements described above relative to the computer 610, although only a memory storage device 681 has been illustrated in FIG. 7. The logical connections depicted in FIG. 7 include a local area network (LAN) 671 and a wide area network (WAN) 673, but may also include other networks. Such networking environments are commonplace in offices, enterprise-wide computer networks, intranets and the Internet.

When used in a LAN networking environment, the computer 610 is connected to the LAN 671 through a network interface or adapter 670. When used in a WAN networking environment, the computer 610 typically includes a modem 672 or other means for establishing communications over the WAN 673, such as the Internet. The modem 672, which may be internal or external, may be connected to the system bus 621 via the user input interface 660, or other appropriate mechanism. In a networked environment, program modules depicted relative to the computer 610, or portions thereof, may be stored in the remote memory storage device. By way of example, and not limitation, FIG. 7 illustrates remote application programs 685 as residing on memory device 681.

The communications connections 670 and 672 allow the device to communicate with other devices. The communications connections 670 and 672 are an example of communication media. The communication media typically embodies computer readable instructions, data structures, program modules or other data in a modulated data signal such as a carrier wave or other transport mechanism and includes any information delivery media. A “modulated data signal” may be a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Computer readable media may include both storage media and communication media.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

1. A method of monitoring a health status of a patient using a monitoring system comprising a first processor [170], a pair of earbud electrocardiogram (ECG) monitors, defined as a first monitor [102] and a second monitor [102], and each including a respective conductive electrode component [110] characterized by at least one physiological-type sensor, and an electrical signal conversion unit [104] including a third electrode component [143] characterized by a physiological-type sensor; the method comprising the steps of:
receiving, using the conductive electrode component [110] of the first monitor [102] that is configured to be positioned in biometric signal communication with a left ear of the patient, biopotential signals from the left ear of the patient, defined as a first reading [520]; and
receiving, using the conductive electrode component [110] of the second monitor [102] that is configured to be positioned in biometric signal communication with a right ear of the patient, biopotential signals from the right ear of the patient, defined as a second reading [520].

2. The method according to claim 1 further comprising the step of receiving, using the third electrode component [143], biopotential signals from one of a left arm and a hip of the patient.

3. The method according to claim 1 wherein the electrical signal conversion unit [104] further comprises a record button; and wherein the step of receiving the first reading further comprises the step of triggering, using the record button, a recording of the biopotential signals from the left ear of the patient.

4. The method according to claim 1 wherein the first monitor [102] and the second monitor [102] each further comprises a speaker unit; and wherein the method further comprises the steps of:
receiving, using the respective speaker unit of each of the first monitor [102] and the second monitor [102], an audio signal; and
converting, using the respective speaker units of the first monitor [102] and the second monitor [102], the audio signal into sound.

5. The method according to claim 4 wherein the electrical signal conversion unit [104] further comprises a slide switch; and wherein the step of converting the audio signal further comprises the step of altering, using the slide switch, a volume of the sound.

6. The method according to claim 1 further comprising the steps of:
receiving, using the electrical signal conversion unit [104], at least one of the first reading and the second reading, to define a received signal; and
converting, using the electrical signal conversion unit [104], the received signal into converted ECG data [530].

7. The method according to claim 6 wherein the first processor [170] further comprises a smartphone [170] characterized by at least one application; and wherein the method further comprises the steps of:
receiving, using the smartphone [170], the converted ECG data, and
analyzing, using the at least one application of the smartphone [170], the converted ECG data for cardiovascular health indicators [540].

8. The method according to claim 6 wherein the electrical signal conversion unit [104] further comprises a system on a chip (SoC) [200] that includes a data store [240] and a second processor [250]; and wherein converting the received signal into converted ECG data [530] further comprises:
retrieving, using the second processor [250], processing functions from the data store [240]; and
executing, using the second processor [250], the processing functions to identify the health status of the patient from at least one of the first reading and the second reading.

9. The method according to claim 8 wherein the SoC [200] is configured in data communication with the smartphone [170]; and wherein the method further comprises the steps of
retrieving, using the second processor [250], transmission instructions from the data store [240]; and
executing, using the second processor [250], the transmission instructions to transmit the health status of the patient to the smartphone [170].

10. A monitoring system [100] comprising:
a first processor [170];
a pair of earbud electrocardiogram (ECG) monitors, defined as a first monitor [102] and a second monitor [102], and each including a respective conductive electrode component [110] characterized by at least one physiological-type sensor; and
an electrical signal conversion unit [104] including a third electrode component [143] characterized by a physiological-type sensor;
wherein the conductive electrode component [110] of the first monitor [102] is configured to be positioned in biometric signal communication [515] with a left ear of the patient, and to receive biopotential signals from the left ear of the patient, defined as a first reading [520];
wherein the conductive electrode component [110] of the second monitor [102] is configured to be positioned in biometric signal communication [515] with a right ear of the patient, and to receive biopotential signals from the right ear of the patient, defined as a second reading [520].

11. The monitoring system [100] according to claim 10 wherein the third electrode component [143] is configured to receive biopotential signals from one of a left arm and a hip of the patient.

12. The monitoring system [100] according to claim 10 wherein the electrical signal conversion unit [104] further comprises a record button; configured to trigger a recording of the biopotential signals from the left ear of the patient.

13. The monitoring system [100] according to claim 10 wherein the first monitor [102] and the second monitor [102] each further comprises a speaker unit configured to receive an audio signal and to convert the audio signal into sound.

14. The monitoring system [100] according to claim 10 wherein the electrical signal conversion unit [104] further comprises a system on a chip (SoC) [200].

15. The monitoring system [100] according to claim 14 wherein the SoC [200] further comprises a data store [240] and a second processor [250], wherein the second processor [250] is configured to retrieve processing functions from the data store [240], wherein the processing functions, when executed by the second processor [250], are configured to identify a health status of the patient from at least one of the first reading [520] and the second reading [520].

16. The monitoring system [100] according to claim 14 wherein the first processor [170] further comprises a smartphone [170]; and wherein the SoC [200] of the electrical signal conversion unit [104] is configured in wired data communication with the smartphone [170].

17. The monitoring system [100] according to claim 14 wherein the first processor [170] further comprises a smartphone [170]; and wherein the SoC [200] of the electrical signal conversion unit [104] is configured in wireless data communication with the smartphone [170].

18. The monitoring system [100] according to claim 17 wherein the SOC [200] of the electrical signal conversion unit [104] further comprises a transmitter [260] configured in wireless data communication with the smartphone [170]; and wherein the processing functions, when executed by the second processor [250], are further configured to write the health status of the patient to the transmitter [260].

19. The monitoring system [100] according to claim 17 wherein the SOC [200] of the electrical signal conversion unit [104] further comprises a receiver [270] configured in wireless data communication with the smartphone [170]; wherein the processing functions, when executed by the second processor [250], are further configured to read data from the receiver [270].

20. The monitoring system [100] according to claim 14 wherein the SoC [200] of the electrical signal conversion unit [104] further comprises a power supply [280] characterized by a thin-film thermoelectric power generator configured to harvest electrical energy produced as body heat by subcutaneous tissue of the patient.
